(19) United States
(12) Patent Application Publication
(10) Pub. No.: US 2007/0185479 A1
(43) Pub. Date: Aug. 9, 2007

(54) Methods and devices for performing ablation and assessing efficacy thereof

(76) Inventor: Liming Lau, Mountain View, CA (US)

Correspondence Address: LAW OFFICE OF ALAN W. CANNON, 942 MESA OAK COURT, SUNNYVALE, CA, 94086, US

(21) Appl. No.: 11/348,177

(22) Filed: Feb. 6, 2006

(51) Int. Cl. A61B 18/18

(52) U.S. Cl. 606/33; 606/41

(57) ABSTRACT

Devices and methods for performing an ablation procedure. One embodiment of a device includes a main body having proximal and distal end portions and first and second sides extending between the proximal and distal end portions. At least one ablation element is configured to apply an ablation energy or substance to tissue to be ablated. At least one electrode is movably positioned with respect to at least one of the sides of the main body, and each such electrode may be configured to assume a retracted position along that respective side of the main body, and an extended position extending beyond a perimeter of the main body. In one method embodiment, a method of performing ablation includes delivering an ablation energy or substance from an ablation device to a target area of tissue to form a lesion therein; delivering an electrical signal via said ablation device to non-ablated tissue on one side of the lesion; and monitoring non-ablated tissue on an opposite side of the lesion to assess whether electrical conduction through the lesion has occurred.

### FIELD OF THE INVENTION

The present invention applies to the field of devices and methods for performing surgery, more particularly to devices and methods for performing and monitoring ablation procedures.

### BACKGROUND OF THE INVENTION

Various medical conditions, diseases and dysfunctions may be treated by ablation, using various ablation devices and techniques. Ablation is generally carried out to kill or destroy tissue at the site of treatment to bring about an improvement in the medical condition being treated.

In the cardiac field, aberrant signals in the heart can cause arrhythmias such as atrial fibrillation and flutter, and these are conditions that have been treated with some success by various procedures using many different types of ablation technologies to interrupt conduction of the aberrant signals. Atrial fibrillation continues to be one of the most persistent and common of the cardiac arrhythmias, and may further be associated with other cardiovascular conditions such as stroke, congestive heart failure, cardiac arrest, and/or hypertensive cardiovascular disease, among others. Left untreated, serious consequences may result from atrial fibrillation, whether or not associated with the other conditions mentioned, including reduced cardiac output and other hemodynamic consequences due to a loss of coordination and synchronicity of the beating of the atria and the ventricles, possible irregular ventricular rhythm, atrioventricular valve regurgitation, and increased risk of thromboembolism and stroke.

Various procedures and technologies have been applied to the treatment of atrial arrhythmias/fibrillation. Drug treatment is often the first approach to treatment, where it is attempted to maintain normal sinus rhythm and/or decrease ventricular rhythm. However, drug treatment is often not sufficiently effective and further measures must be taken to control the arrhythmia.

Electrical cardioversion and sometimes chemical cardioversion have been used, with less than satisfactory results, particularly with regard to restoring normal cardiac rhythms and the normal hemodynamics associated with such.

A surgical procedure known as the “MAZE III” or “Cox MAZE III” (which evolved from the original MAZE procedure) procedure involves breaking up real or potential re-entrant circuits (thought to be the drivers of the fibrillation and flutter) by surgically cutting a maze pattern in the atrium to eliminate ectopic foci and to prevent the reentrant circuits from being able to conduct therethrough. The maze pattern according to which the cuts are made may be developed by electrophysiological mapping of the atria to identify macroreentrant circuits and locations of ectopic foci (e.g., non-SA node triggers), or may rely upon mapping research and previously well-established patterns along which to perform ablation. The prevention of the reentrant circuits and signals from ectopic foci allows sinus impulses to activate the atrial myocardium without interference by reentering conduction circuits and signals from ectopic foci, thereby preventing fibrillation and flutter. This procedure has been shown to be effective, but generally requires the use of cardiopulmonary bypass, and is a highly invasive procedure associated with high morbidity.

Other procedures have been developed to perform transmural ablation of the heart wall or adjacent tissue walls. Transmural ablation may be grouped into two main categories of procedures: endocardial and epicardial. Endocardial procedures are performed from inside the wall (typically the myocardium) that is to be ablated, and is generally carried out by delivering one or more ablation devices into the chambers of the heart by catheter delivery, typically through the arteries and/or veins of the patient. Alternatively, endocardial procedure may be performed surgically, such as in the original Cox-Maze procedures. Epicardial procedures are performed from the outside wall (typically the myocardium) of the tissue that is to be ablated, often using devices that are introduced through the chest and between the pericardium and the tissue to be ablated, and access for such introduction may be surgically, or by less invasive, percutaneous techniques. However, mapping may still be required to determine where to apply an epicardial device, which may be accomplished using one or more instruments endocardially, or epicardial mapping may be performed. Various types of ablation devices are provided for both endocardial and epicardial procedures, including radiofrequency (RF), microwave, ultrasound, heated fluids, cryogenics, chemicals and laser. Epicardial ablation techniques provide the distinct advantage that they may be performed on the beating heart without the use of cardiopulmonary bypass.

When performing ablation, a lesion is formed in the tissue by ablation that becomes scar tissue, which does not conduct electrical signals therethrough. Thus, a carefully placed lesion or pattern of lesions can be formed to effectively eliminate signals not originating at the SA (sino-atrial) node and prevent re-entrant circuits or other aberrant signal pathways that cause arrhythmias, flutter or other abnormal heart beat patterns. The formation of lesions must be carefully controlled to ensure that sufficient energy is applied to form a lesion completely through the wall of a tissue to form a conduction block, while on the other hand, ensuring that too much energy is not applied and/or application is not applied over too long a time period, as serious damage to adjacent tissues may result. Currently available surgical ablation devices do not provide a means for assessing the electrophysiological effect of the resulting lesion, which can be useful in determining ablation efficacy.

Thus, there is a continuing need for devices, techniques, systems and procedures for forming lesions in accurate, intended locations, that are sufficiently transmural and continuous and which can be readily assessed at the time of forming such lesions.

### SUMMARY OF THE INVENTION

Devices for performing ablation and assessing the efficacy thereof are disclosed. In one embodiment, a main body having proximal and distal end portions and first and second sides extending between the proximal and distal end portions is provided. At least one ablation element is configured to apply an ablation energy or substance to tissue to be ablated. At least one electrode is movably positioned with respect to at least one of the sides of the main body, and each such electrode may be configured to assume a retracted position along the respective side of the main body, and an extended position extending beyond a perimeter of the main body.

A method for performing ablation as described herein includes delivering an ablation energy or substance from an ablation device to a target area of tissue to form a lesion therein; delivering an electrical signal via said ablation device to non-ablated tissue on one side of the lesion; and monitoring non-ablated tissue on an opposite side of the lesion to assess whether electrical conduction through the lesion has occurred.

A method of assessing the efficacy of an ablation procedure includes contacting an electrode, extending from a device used to perform an ablation, to non-ablated tissue on one side of a target area for lesion formation by the ablation procedure; delivering an electrical signal via said electrode to the non-ablated tissue contacted by the electrode; and monitoring non-ablated tissue on an opposite side of the target area to assess electrical conduction from the electrode to the non-ablated tissue on the opposite side of the target area.

A method of performing atrial ablation is provided to include: delivering an ablation energy or substance from an ablation device to a target area of tissue to form one or more lesions to surround one or more pulmonary vein ostia; delivering an electrical signal via said ablation device to non-ablated tissue on one side of the lesion formation made by said one or more lesions; and monitoring non-ablated tissue on an opposite side of the lesion formation to assess whether electrical conduction across at least one of the lesions, or through a gap between lesions has occurred.

These and other advantages and features of the invention will become apparent to those persons skilled in the art upon reading the details of the devices and methods as more fully described below.

### BRIEF DESCRIPTION OF THE DRAWINGS

FIG. 1 illustrates a sectional view of a myocardial wall in which ablation has been performed to create a lesion to attempt to create an electrical conduction block at the site of lesion.

FIG. 2 is a sectional illustration of a heart with ostia of the pulmonary veins shown, exemplifying a procedure that is typically carried out to treat atrial fibrillation.

FIG. 3 illustrates one example of a device configured to perform ablation, as well as assess the efficacy of a lesion performed during the ablation.

FIG. 4 is a schematic view illustrating an arrangement of channels and openings in the main body of an ablation device.

FIG. 5 is a cross-sectional view of the device of FIG. 3 taken along line 55-55.

FIG. 6A is a sectional illustration of an embodiment wherein wires and channel slots are configured and oriented to direct the extension of wires in predetermined directions.

FIG. 6B illustrates the extension of electrodes outwardly from the perimeter of the main body of an ablation device, to an extent sufficient to contact non-ablated tissue on opposite sides of a lesion formed by the ablation device.

FIG. 7 schematically illustrates a sectional view of an ablation device, with electrodes extending from the sides thereof to contact non-ablated tissue.

FIG. 8A is a sectional view of an ablation device with an electrode extended.

FIG. 8B is a sectional view similar to FIG. 8, but with the electrode retracted.

FIG. 9 is an illustration of a partial view of an ablation device provided with only one channel on one side of the device, with an opening from which an electrode is extended.

FIG. 10 is a sectional partial view illustration of an ablation device having multiple electrodes.

FIG. 11 is a sectional partial view of another ablation device having multiple electrodes.

### DETAILED DESCRIPTION OF THE INVENTION

Before the present devices and methods are described, it is to be understood that this invention is not limited to particular devices and method steps described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a lesion” includes a plurality of such lesions and reference to “the electrode” includes reference to one or more electrodes and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Definitions

The term “ablation” refers to a procedure carried out to kill or destroy tissue at a site of treatment to bring about an improvement in the medical condition being treated. Ablation may be performed by a variety of devices that use varying energy sources or substances to apply to the tissue to be ablated. Examples of such energy sources and substances include, but are not limited to RF energy, cryogenic energy, microwave energy, thermal energy, electrical energy, ultrasound and chemical substances.

The term “endocardial ablation” refers to ablation performed by application of ablation energy or ablation substance from inside of the cardiac tissue, e.g., to the inner wall of the myocardium or other tissue.

The term “epicardial ablation” refers to ablation performed by application of ablation energy or ablation substance from outside the cardiac tissue, e.g. to the outer wall of the myocardium or other tissue.

The term “open-chest procedure” refers to a surgical procedure wherein access for performing the procedure is provided by a full sternotomy or thoracotomy with rib spreading, a sternotomy wherein the sternum is incised and the cut sternum is separated using a sternal retractor, or a thoracotomy wherein an incision is performed between a patient's ribs and the incision between the ribs is separated using a retractor to open the chest cavity for access thereto.

The term “closed-chest procedure” or “minimally invasive procedure” refers to a surgical procedure wherein access for performing the procedure is provided by one or more openings which are much smaller than the opening provided by an open-chest procedure, and wherein a traditional sternotomy is not performed. Closed-chest or minimally invasive procedures may include those where access is provided by any of a number of different approaches, including mini-sternotomy, thoracotomy or mini-thoracotomy, or less invasively through a port provided within the chest cavity of the patient, e.g., between the ribs or in a subxyphoid area, with or without the visual assistance of a thoracoscope.

The term “reduced-access surgical site” refers to a surgical site or operating space that has not been opened fully to the environment for access by a surgeon. Thus, for example, closed-chest procedures are carried out in reduced-access surgical sites. Other procedures, including procedures outside of the chest cavity, such as in the abdominal cavity or other locations of the body, may be carried out as reduced access procedures in reduced-access surgical sites. For example, the surgical site may be accessed through one or more ports, cannulae, or other small opening(s). What is often referred to as endoscopic surgery is surgery carried out in a reduced-access surgical site.

Devices and Methods

The following devices described are for performing ablation in a controlled manner, in such a way that the operator/surgeon can readily determine an amount of ablation that has occurred, using features of the same device used to perform the ablation. The present invention may include devices for performing ablation in any surgical environment, including open surgical environments, such as an open-chest environment or other surgical site that has been opened for direct access during the procedure, or reduced access surgical sites, including closed chest cardiac procedures or other surgical sites that are accessible only though small ports or intravascularly.

The present invention is not limited to the source of ablation energy or substance used to perform the ablation, and may include microwave, RF, ultrasound, cryogenic gas, thermal or chemical ablation source, for example. Further the present invention is not limited to any particular device configuration, such as to length, rigidity, flexibility, malleability, width, etc., as the principles of the present invention apply to variations in such characteristics of ablation devices.

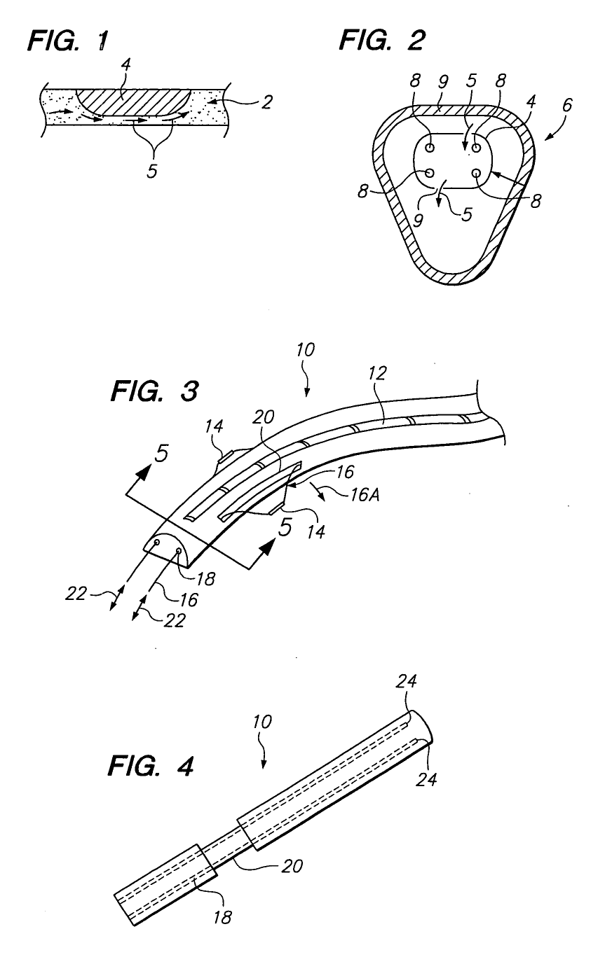

As noted above, when performing ablation, it is desirable and often times necessary to form a lesion that completely traverses the wall of the tissue being ablated. This is necessary when ablation is performed to create an electrical conduction block, such as in the case of myocardial ablation for example. FIG. 1 illustrates a sectional view of a myocardial wall 2 in which ablation has been performed to create a lesion 4 to attempt to create an electrical conduction block at the site of lesion 4, such as for example, in the performance of a Cox-Maze type lesion pattern to treat atrial fibrillation. In this example, however, ablation energy has not been applied significantly to establish the lesion 4 to completely traverse the tissue wall 2. As a result, electrical signals are still able to pass across the location of the lesion in the location of the tissue that has not been ablated, as indicated by arrows 5 in FIG. 1. In such instances, when one or more lesions of a lesion set performed by an ablation procedure is incomplete, the lesions created may be insufficient to effectively interrupt electrical conduction through the ablated tissue to effectively treat the condition for which the procedure was performed. For example, in this case, the atrial fibrillation may not adequately abate.

FIG. 2 is a sectional illustration of a heart 6 with ostia 8 of the pulmonary veins shown, exemplifying another procedure that is typically carried out to treat atrial fibrillation. As at least part of this procedure, a “box formation” of lesions 4 is formed to surround the ostia 8 to prevent signals thought to emanate from the pulmonary veins, thereby preventing them from entering the atrial myocardial tissue and preventing atrial fibrillation caused thereby. Additionally, or alternatively, ablation of ganglionated plexi may be ablated or the box formation may be formed to also surround such ganglionated plexi, as it is hypothesized that these nerve endings may trigger the release of certain hormones to cause a sympathetic or parasympathetic response in the cardiac tissue. FIG. 2 illustrates another potential failure mode in that if the lesions 4 are not continuously joined to completely encircle the pulmonary vein ostia 8, then electrical signals may still be able to conduct through any gap or discontinuity 9 in the box as indicated by arrows 5. Thus, all lesions 4 must pass entirely through the wall of the myocardium or be acutely non-transmural but forming resultant fibrosis/scarring to effectively render a fully transmural conduction block or to at least sufficiently disrupt electrical conduction to an extent sufficient to prevent beating of the cardiac tissue, and the lesions 4 (or at least the resultant fibrosis/scarring resultant from lesion formations) must also continuously join one another to sufficiently disrupt electrical conduction block (such as determined by an electrophysiologist, for example) or to form a complete conduction block, electrically separating the ostia 8 from the myocardium of the atrium. To assess electrophysiological efficacy, a surgeon would heretofore use a separate pacing/electrophysiological system to perform a mapping/conduction procedure to determine whether a complete blockage has been achieved by the lesions formed. This is inconvenient, requires additional time to complete the procedure and requires additional equipment to be handled.

FIG. 3 illustrates one example of a device 10 configured to perform ablation, as well as assess the efficacy of the lesion performed during the ablation. One or more ablation elements or effectors may be mounted on device 10 at locations 12 to apply the ablation energy or substance to the tissue to perform the ablation. Alternatively, an ablation element may be slidably positioned within the body of the device 10 and aligned by a user at a desired segment 12 to properly position the ablation element in a location where a lesion is desired to be formed. In the example shown, a series of such segments 12 are provided in a linear array, such that lesions can be formed incrementally by repetitively positioning an ablation element at different segments and applying ablation energy at each segment location where it is desired to form a lesion. In this way, a series of lesions can be sequentially formed, and such lesions may be formed to overlap one another to form an extended lesion through (or sufficiently through, to an extent described above) tissue that is applied to. An example of an ablation device so configured is the Flex 10 microwave probe (Guidant Corporation, Santa Clara, Calif.), although the present invention is not limited to application to this product only, but may be configured in any ablation device, as noted previously.

Mapping/pacing electrodes 14 are provided along the sides or periphery of device 10 and are actuatable to be positioned on opposite sides of a lesion to test the efficacy of a lesion that has been formed by ablation element 12 in a manner as described below. Each electrode 14 is mounted to a push wire 16 that is slidably mounted in a lumen or channel 18 in the body of device 10. Push wire 16 may be electrically conductive and, in this case, electrode 14 is electrically connected to push wire 16 so that an electrical signal can be supplied from a power source proximal of device 10 for delivery to electrode 14. Alternatively, an additional electrically conductive lead (not shown) may be electrically connected to electrode 14 and run alongside of push wire 18, proximally out of device 10 to be connected to a power source and/or monitor.

A slot, notch or opening 20 is formed in the body of device 10 adjacent each electrode 14, so that electrode 14 may be extended outwardly from the body of device 10 to extend beyond the perimeter of the device body, as shown in FIG. 3. Slot, notch or opening 20 typically extends beyond the location of electrode 14 both distally and proximally, to allow push wire 16 to flex outwardly when a operator pushes on push wire 16 from a location proximal of the proximal end of device 10, as indicated by arrow 22. The distal end of each lumen or channel 18 is closed 24, as shown in FIG. 4, so as to function as a stop against which the distal end of a push wire 16 inserted therein abuts as the operator pushes on push wire 16. Once so abutted, continued pushing on push wire 16 causes it to flex outwardly with sufficient extension to place electrode 14 in a location beyond the extent of the ablated tissue, in any areas where it is not constrained by channel 18, i.e., at the location of opening 20.

Push wire 16 may be fixed at its distal end in channel 18, such as by crimping, adhesives, welding or the like, to permanently fix each push wire in device 10. Alternatively, distal ends of push wires 16 may simply physically abut against the distal ends of respective channels 18 to accomplish the flexing described, and in this case, push wires 16 and electrodes 14 are removable from device 10. In this case, push wires/electrodes may be configured as an optional feature for device 10 and separately provided, so that the user has the option of whether to include this feature when performing an ablation procedure.

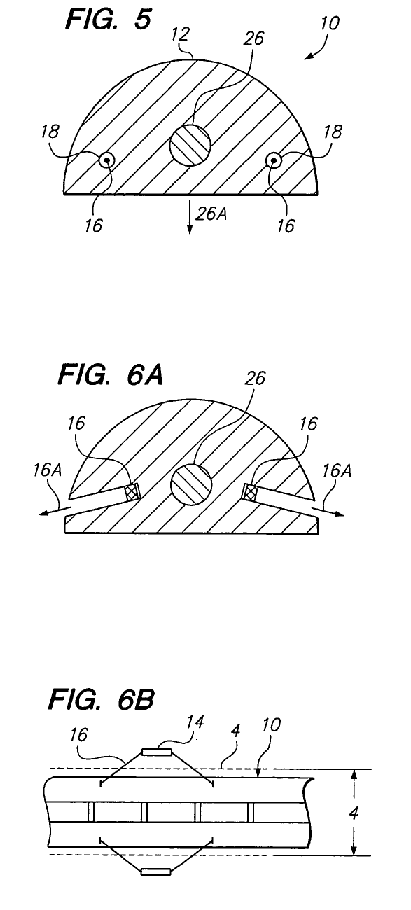

As shown in FIG. 3, an electrode 14 and associated push wire 16 are provided on two sides of device 10. Electrode 14/wire 16 on one side of device 10 are electrically independent of electrode 14/wire 16 on the opposite side of device 10 and are capable of independently reading/transmitting signals on opposite sides of device 10. Also, the wires 16 on opposite sides of the device may be mechanically independent, so as to be actuated/extended independently of one another. Alternatively, wires 16 may be mechanically linked to allow extension of wires 16 on opposite sides with a single mechanical actuation. In use, ablation elements 12 are contacted against the tissue where an ablation is desired to be performed (either on an inner wall or outer wall of tissue, as described previously). In the case of FIG. 3, microwave energy is applied via an ablation element, sequentially at the desired segment locations 12, although the following procedure is applicable to lesions formed by application of any type of ablation energy or substance. In this example, however, microwave energy is applied through an ablation element sequentially at selected segment 12 to form a lesion in the tissue at each selected segment 12 in the locations along the tissue that these segments are adjacent to. The energy may be applied sequentially device 10 at locations indicated by through each of the segments 12 shown, in direction 26A, or according to any other selection pattern as directed through antenna 26 (see cross-sectional view of FIG. 5). Once the desired lesion has been formed, the operator/surgeon discontinues application of ablation energy to the tissue and pushes on push wires 16 to extend electrodes 14 outwardly from the perimeter of the body of device 10, as shown in FIG. 3. Push wires 16 are configured to flex in the direction of arrow 16A (i.e., toward the tissue surface) to ensure that the electrodes contact the tissue upon extension. Wires 16 may be pre-shaped to extend in this direction, biased to extend in this direction, and/or be oriented with respect to device 10 to extend in this direction. This allows the user to leave device 10 in its current position relative to the tissue as testing with electrode 14 is being conducted. In this way, if it is determined that one or more locations need additional ablation, as indicated by signal conduction from one side of device 10 to the other side (as monitored through electrodes/wires 14/16), then additional ablating energy can be reapplied through the same location or locations that the energy was originally applied, to further extend the lesion or lesions.

FIG. 6A is a sectional illustration according to one embodiment wherein wires 16 and channel slots 20 are configured and oriented to direct the extension of wires 16 (and electrodes 14) in the directions 16A as indicated by the arrows in FIG. 6A. In this example, wires 16 are configured as flattened ribbons so that they bend preferentially about one axis of bending when they are pushed on. Additionally, channel slots are angled and aligned with the direction in which wires 14 are oriented to bend, so that wires 16 and electrodes 14 are guided outwardly and downwardly in the directions of arrows 16A when wires 16 are pushed on from a proximal end portion of device 10.

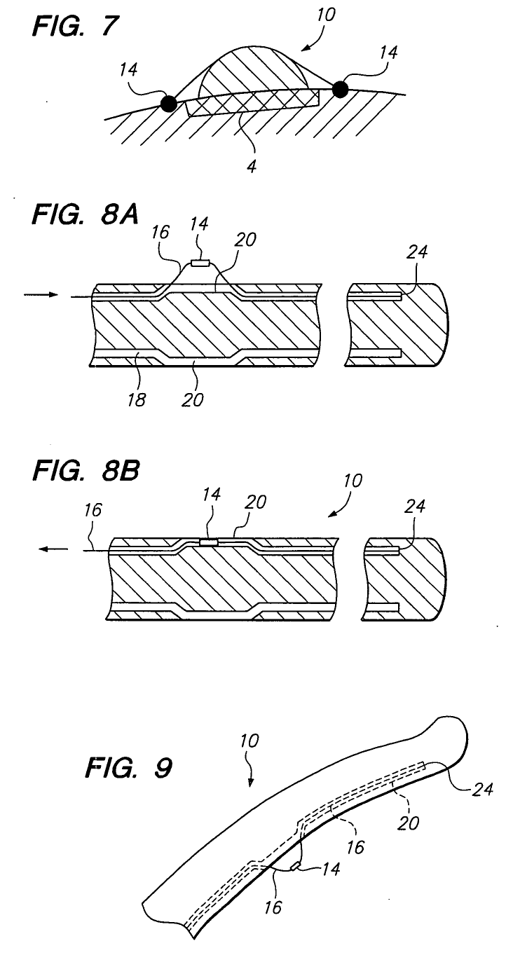

Electrodes 14 are extended sufficiently to contact non-ablated tissue on opposite sides of lesion 4, as shown in FIG. 6B, in preparation for assessment of the electrical conduction properties across the lesion. Note that the width of lesion 4 can be and often is wider than the width of device 10. FIG. 7 is a schematic, sectional view showing electrodes 14 contacting the non-ablated tissue on opposite sides of lesion 4, in the manner described. Using standard electrophysiology equipment including a power source and monitor, an electrical signal, such as an impulse, series of impulses, or other predefined electrical signal having known characteristics suitable for stimulating the tissue to contract (e.g., a known pacing signal may be inputted in the case of cardiac tissue) is inputted to one of the electrodes 14 on one side of the lesion. The other electrode 14, on the opposite side of the lesion is electrically connected to the monitoring equipment to determine whether the signal inputted in the first electrode is received by this electrode. If no signal is received, or if a delayed or weakened signal is received that is indicative of the signal passing around the length of the lesion to reach the opposite electrode (as can be confirmed by time delay formula known to electrophysiologists), then it can be confirmed that the lesion is blocking conduction and it can be concluded that the lesion has been fully formed through the wall of tissue. The monitoring equipment may display the signal inputted by the sending electrode 14 as well as that received by one or more receiving electrodes 14 as well, as such monitors are currently available.

On the other hand, if a signal is received sooner than that described, this would be indicative that the signal is passing across the location of the lesion and that therefore the lesion has not been sufficiently formed. Reapplication of ablation energy would then be performed, and the lesion would be re-tested according to the procedure just described. Advantageously, device 10 may be maintained in the targeted position for performing the ablation during the testing procedure, thereby saving time and effort if a reapplication of ablation energy/substance is required after testing, since device 10 does not need to be repositioned in such an event. Also, this improves accuracy of the placement of the reapplication of the ablation energy, since device 10 will not have been moved since the previous application of ablation energy. Reiteration of ablation energy and testing may be performed until it has been determined that the lesion has been fully formed so as to function as an effective conduction block.

In the case where a box lesion is formed, as described above with regard to FIG. 2, such as by encircling the pulmonary vein ostia 8 with the flexible device 10 of FIG. 3, for example, and forming a lesion to completely encircle the pulmonary vein ostia 8, application of a signal through one of the electrodes 14 should not be received by the opposite electrode 14, (or should at least be disrupted so as to be incapable of triggering beating of the cardiac tissue) since the tissue on one side of the lesion should be sufficiently isolated from the tissue on the other side. Thus, testing will confirm that the box lesion 4 has been completely and successfully formed when, upon testing, the test signal is not received by the opposite electrode 14, or a signal that is received is determined, by monitoring, to be insufficient to cause contraction of the cardiac tissue to cause the fibrillation or other condition being treated.

FIG. 8A is a partial sectional view showing a configuration of device 10 with electrode 14 extended through the action of pushing on push wire 16 in the direction of the arrow shown. Note that in this example, slot, opening or cavity 20 is offset from the remainder of channel 18 so that push wire 16 is slightly bent or curved in an outward direction, even when electrode 14 is retracted as shown in FIG. 8B. This helps assure that push wire 16 bends outwardly when an operator pushes on push wire 16 from a proximal location, to ensure that electrode is positioned outside of the periphery of the main body of device 10. When the push wire 16 positioned as shown in FIG. 8A is then pulled by an operator in the direction of the arrow shown in FIG. 8B, push wire 16 becomes relatively straight, thereby retracting electrode 14 to a position in opening 20, within the confines of the perimeter of the body of device 10.

Alternative to the configurations already described, a device 10 may be provided with an electrode 14 on only one side of device 10. This may be accomplished, in the embodiments with removable push wires 16, by including only one push wire 16 and associated electrode 14 in one channel 18, while leaving the other channel empty. Alternatively, a device 10 such as shown in FIG. 9 may be provided with only one channel 18, in which a push wire 16/electrode 14 assembly may be either permanently or removably housed in manners already described. With such an arrangement, electrode 14 can be extended, after at least partial formation of a lesion 4 to contact non-ablated tissue outside of the perimeter of lesion 4, on one side of it. Upon application of a signal to the non-ablated tissue (such as a pacing signal for example), the operator/surgeon then visually observes the non-ablated tissue on the opposite side of the lesion 4 to see if it contracts or reacts in any way. Such observation may be direct observation, in the case of an open surgical site, or by means of an endoscope or other viewing apparatus in the case of a closed surgical site. Reapplication of ablation energy/substance is carried out in the case that a reaction is seen in the tissue on the opposite side of the lesion 4 from which the signal was inputted via electrode 14.

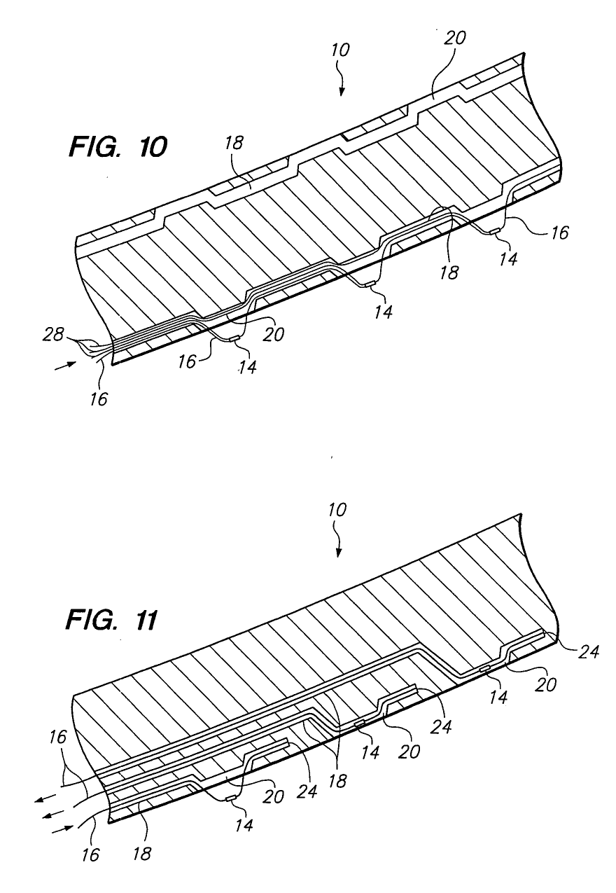

In another arrangement, multiple electrodes 14 may be provided in device 10 on one or both sides of device 10. FIG. 10 shows device 10 configured for multiple electrodes on both sides of the device, wherein electrodes are shown on only one side of the device for simplicity, the electrodes and wires on the opposite side having been removed from channel 18. In this arrangement, all electrodes 14 on one side are connected to a single push wire 16, and multiple openings 20 are provided for extending the electrodes 14 therefrom respectively. Upon pushing on push wire 16 in the direction of the arrow shown, all electrodes 14 connected thereto are extended outwardly from the periphery of the main body of device 10 as shown. Each electrode 14 in this instance is electrically connected to an additional electrical line (electrically conductive lead) 28 so that the electrodes can be independently actuated. In this way, a test or pacing signal can be delivered to a first of the electrodes 14 while not conducting a signal through any of the other electrodes on that side. Testing on the opposite side of the lesion may be performed visually, as described, by monitoring the electrode 14 directly opposite the electrode 14 through which the signal has been delivered, or by monitoring multiple or all of the electrodes 14 on the opposite side. After completion of testing via a signal through a first of the electrodes 14, the same procedure can be conducted through a second of the electrodes on the same side, and so forth, until all electrodes on that side have delivered test or pacing signals. Of course a fewer number than all of the electrodes may be used to deliver testing signals, according to the operator's judgment. It is further noted that both sides may be configured similarly, so that test signals can be sent from either side of device 10 and monitored from the opposite side as desired by the operator. A similar though alternative configuration may be provided in which device 10 is configured with a channel 18 and openings 20 on only one side of the device, wherein visual testing is employed.

FIG. 11 is a partial, sectional view showing another variation of a device employing multiple electrodes 14. Although electrodes 14 are shown on only one side of device 10, another arrangement the same as shown on one side in FIG. 11, may be provided on the other side of device 10 to provide a device 10 with independently controllable, multiple electrodes 14 on both sides of the device. In this arrangement, each electrode is mounted to a separate push wire 16. In this way, a single push wire can be pushed upon to extend a single electrode, as is illustrated in FIG. 11. Also, a signal can be inputted to a single electrode 14 to the push wire that it is attached to, without the need for additional electric conduction lines. Separate channels 18 and stops 24 may be provided through which the push wires may be inserted. Alternatively, all push wires 16 may be inserted through a single channel like that shown in FIG. 10, in which case all push wires would extend to the distal end portion of device 10 to abut against single stop 24.

After testing with a first electrode 14 (and, optionally, one or more electrodes 14 on the opposite side of device 10), the push wire 16 connected to the first electrode 14 may be pulled to retract that electrode, and another electrode may be extended from device 10 by pushing on the push wire 16 connected to it. In this way, electrodes 14 may be sequentially deployed and used for testing. As noted before, all electrodes 14 may be extended and still operated individually and sequentially since they are connected to independent electrical conducting lines (push wire 16). Independent control of the use of electrodes 14 for monitoring is provided in the same way.

Alternative, or additional to any of the testing and monitoring procedures described above which may be performed after initial formation of one or more lesions, it is noted that any of these procedures may also be practiced during ablation. Thus, even during the initial ablation/lesion formation, electrodes 14 may be deployed in any of the manners describe above and used to monitor electrical signals while an ablation is being performed. By monitoring a local electrogram (i.e., local to the location that the lesion is being formed in), this information can be used as feedback to determine when to stop an ablation procedure, such as when conduction is sufficiently blocked, or the delay of a signal, as described above, indicates that signal is not passing across the location of the lesion being monitored, or a sufficiently disrupted signal is being received. Such electrograms may be monitored by a physician, for example, or may be automatically monitored (built-in) by the energy source/generator supplying the monitoring signals, for example.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A device for performing an ablation procedure comprising:
a main body having proximal and distal end portions and first and second sides extending between said proximal and distal end portions;
at least one ablation element configured to apply an ablation energy or substance to tissue to be ablated; and
at least one electrode movably positioned with respect to at least one of said sides, each said electrode configured to assume a retracted position along said respective at least one side, and an extended position extending beyond a perimeter of said main body.

2. The device of claim 1 comprising an array of said ablation elements.

3. The device of claim 1 comprising a plurality of said ablation elements.

4. The device of claim 1, wherein said at least one electrode comprises at least a pair of electrodes, a first electrode of each said pair being positioned along said first side, and a second electrode of each said pair being positioned along said second side, opposite said first electrode, respectively.

5. The device of claim 1, wherein said at least one electrode comprises at least two electrodes, a first electrode of said at least two electrodes being positioned along said first side, and a second electrode of said at least two electrodes being positioned along said second side.

6. The device of claim 1, wherein said main body comprises a channel extending distally therein from an opening in said proximal end portion.

7. The device of claim 6, further comprising a push wire configured to be received in said channel, at least one of said at least one electrodes being mounted on said push wire.

8. The device of claim 7, wherein said push wire is electrically conductive and said at least one electrode is electrically connected to said push wire.

9. The device of claim 7, wherein said push wire is fixed to said main body at a distal end of said channel.

10. The device of claim 7, wherein said push wire is removably received in said channel, a distal end of said channel being closed and providing a stop against which a distal end of said push wire is abuttable against.

11. The device of claim 6, wherein said channel comprises a closed distal end.

12. The device of claim 4, wherein said main body comprises a pair of channels extending distally therein from openings in said proximal end portion, a first channel of said pair extending along and adjacent said first side, and a second channel of said pair extending along and adjacent said second side.

13. The device of claim 12, further comprising a pair of push wires configured to be received in said pair of channels, respectively, at least one of said first electrodes being mounted on a first push wire of said pair of push wires and at least one of said second electrodes being mounted on a second push wire of said pair of push wires.

14. The device of claim 13 comprising a plurality of said pairs of electrodes, each of said first electrodes being mounted on said first push wire and each of said second electrodes being mounted on said second push wire.

15. The device of claim 14, further comprising a plurality of electrically conductive leads, each of said electrodes being electrically connected to one of said electrically conductive leads, respectively, said electrically conductive leads extending proximally out of said main body.

16. The device of claim 13 comprising a plurality of pairs of said push wires, each of said first electrodes being mounted on one of said first push wires, respectively, and each of said second electrodes being mounted on one of said second push wires, respectively, such that only one of said electrodes is mounted on any one of said push wires.

17. The device of claim 16, wherein said push wires are electrically conductive and said electrodes are electrically connected to said push wires.

18. The device of claim 4, wherein said main body comprises a plurality of pairs of channels extending distally therein from openings in said proximal end portion, a first channel of each said pair extending along said first side, and a second channel of each said pair extending along said second side.

19. The device of claim 18, further comprising a plurality of pairs of push wires configured to be received in said plurality of pairs of channels, respectively, said first electrodes being mounted on first push wires of said pairs of push wires and said second electrodes being mounted on second push wires of said pairs of push wires, respectively, wherein only one of said electrodes is mounted on any one of said push wires.

20. The device of claim 19, wherein said push wires are electrically conductive and said electrodes are electrically connected to said push wires.

21. The device of claim 6, further comprising at least one opening in at least one of said first and second sides, connecting with said channel, configured to receive one of said at least one electrodes in said retracted position.

22. A method of performing ablation, said method comprising the steps of:
delivering an ablation energy or substance from an ablation device to a target area of tissue to form a lesion therein;
delivering an electrical signal via said ablation device to non-ablated tissue on one side of the lesion; and
monitoring non-ablated tissue on an opposite side of the lesion to assess whether electrical conduction through the lesion has occurred.

23. The method of claim 22, wherein said monitoring comprises visual monitoring of the non-ablated tissue on the opposite side.

24. The method of claim 22, wherein said monitoring comprises contacting an electrode of said ablation device to the non-ablated tissue on the opposite side and interpreting any receipt of the electrical signal or lack thereof via monitoring equipment electrically connected to said electrode.

25. The method of claim 22, wherein said delivering further comprises extending at least one electrode beyond a perimeter of a main body of said ablation device to extend over the lesion and contact the non-ablated tissue, wherein said at least one electrode is electrically connected to a power source.

26. The method of claim 25, further comprising retracting said at least one electrode after a determination that no electrical conduction through the lesion has occurred.

27. The method of claim 22, further comprising re-applying the ablation energy or substance from said ablation device when said monitoring has determined that conduction across the lesion has occurred.

28. The method of claim 27, further comprising repeating said delivering and monitoring steps to assess whether electrical conduction through the lesion has occurred, and repeating said re-applying, delivering and monitoring steps if electrical conduction through the lesion continues.

29. The method of claim 22, wherein said delivering an electrical signal and said monitoring are carried out concurrently with said delivering an ablation energy or substance, said method further comprising ceasing said delivering an ablation energy or substance when it is determined by said monitoring that the lesion has formed sufficiently to create a conduction block.

30. The method of claim 29, wherein said monitoring comprises monitoring an electrogram of said electrical signal as received on the opposite side.

31. The method of claim 22, wherein said delivering an electrical signal and said monitoring are carried out after ceasing said delivering an ablation energy or substance, said method further comprising re-applying the ablation energy or substance from said ablation device when said monitoring has determined that blocking of conduction across the lesion is insufficient.

32. A method of assessing the efficacy of an ablation procedure, said method comprising the steps of:
contacting an electrode, extending from a device used to perform an ablation, to non-ablated tissue on one side of a target area for lesion formation by the ablation procedure;
delivering an electrical signal via said electrode to the non-ablated tissue contacted by the electrode; and
monitoring non-ablated tissue on an opposite side of the target area to assess electrical conduction from said electrode to said non-ablated tissue on the opposite side of the target area.

33. The method of claim 32, wherein said monitoring comprises visual monitoring of the non-ablated tissue on the opposite side.

34. The method of claim 32, wherein said monitoring comprises contacting a second electrode, extending from said ablation device on a side opposite of said electrode delivering the electrical signal, to the non-ablated tissue on the opposite side and interpreting any receipt of the electrical signal or lack thereof via monitoring equipment electrically connected to said second electrode.

35. The method of claim 32, further comprising applying ablation energy or substance from said ablation device when said monitoring has determined that electrical conduction across the target area has occurred.

36. The method of claim 35, wherein said ablation device remains in position for application of ablation energy or substance during said contacting, delivering and monitoring steps, so that said ablation device does not need to be repositioned for said applying ablation energy.

37. The method of claim 35, further comprising repeating said delivering and monitoring steps to assess whether electrical conduction through the lesion formed at the target area has occurred, and repeating said applying, delivering and monitoring steps if electrical conduction through the lesion continues.

38. The method of claim 32, further comprising applying ablation energy or substance from said ablation device to the target area during said delivering and monitoring, said method further comprising ceasing said applying ablation energy or substance when it is determined by said monitoring that the lesion has formed sufficiently.

39. The method of claim 38, wherein said monitoring comprises monitoring an electrogram of said electrical signal as received on the opposite side.

40. A method of performing atrial ablation, said method comprising the steps of:
delivering an ablation energy or substance from an ablation device to a target area of tissue to form one or more lesions to surround one or more pulmonary vein ostia;
delivering an electrical signal via said ablation device to non-ablated tissue on one side of the lesion formation made by said one or more lesions; and
monitoring non-ablated tissue on an opposite side of the lesion formation to assess whether electrical conduction across at least one of the lesions, or through a gap between lesions has occurred.

41. The method of claim 40, wherein said monitoring comprises visual monitoring of the non-ablated tissue on the opposite side.

42. The method of claim 40, wherein said monitoring comprises contacting a second electrode, extending from said ablation device on a side opposite of said electrode delivering the electrical signal, to the non-ablated tissue on the opposite side and interpreting any receipt of the electrical signal or lack thereof via monitoring equipment electrically connected to said second electrode.

43. The method of claim 40, further comprising re-applying ablation energy or substance from said ablation device when said monitoring has determined that electrical conduction has occurred to the opposite side.

44. The method of claim 40, wherein said delivering an electrical signal and said monitoring are carried out concurrently with said delivering an ablation energy or substance, said method further comprising ceasing said delivering an ablation energy or substance when it is determined by said monitoring that the lesion has formed sufficiently.

45. The method of claim 44, wherein said monitoring comprises monitoring an electrogram of said electrical signal as received on the opposite side.

46. The method of claim 40, wherein said delivering an electrical signal and said monitoring are carried out after cessation of said delivering an ablation energy or substance, said method further comprising reapplying said delivering an ablation energy or substance when it is determined by said monitoring that the lesion has not formed sufficiently.
